(19) United States
(12) Patent Application Publication
(10) Pub. No.: US 2010/0226937 A1
(43) Pub. Date: Sep. 9, 2010

(54) COMBINATION VACCINES WITH 1-HYDROXY-2-PHENOXYETHANE PRESERVATIVE

(75) Inventor: Mario Contorni, Siena (IT)

Correspondence Address: NOVARTIS VACCINES AND DIAGNOSTICS INC., INTELLECTUAL PROPERTY- X100B, P.O. BOX 8097, Emeryville, CA, 94662-8097, US

(73) Assignee: NOVARTIS AG, Basel (CH)

(21) Appl. No.: 12/377,370

(22) PCT Filed: Aug. 15, 2007
(86) PCT No.: PCT/IB2007/003211

§ 371 Date: Feb. 12, 2009

(30) Foreign Application Priority Data
Aug. 15, 2006 (GB) 0616226.7

(51) Int. Cl. A61K 39/295; A61K 39/116; A61P 31/04; A61P 31/12

(52) U.S. Cl. 424/201.1; 424/203.1

(57) ABSTRACT

Processes for preparing combination vaccines that include diphtheria and tetanus toxoids, where these two toxoids are used in the processes as a single component containing both toxoids, and also containing 1-hydroxy-2-phenoxyethane.

All documents cited herein are incorporated by reference in their entirety.

### TECHNICAL FIELD

This invention is in the field of manufacturing combination vaccines, that is vaccines containing mixed immunogens from more than one pathogen, such that administration of the vaccine can simultaneously immunize a subject against more than one pathogen.

### BACKGROUND ART

Vaccines containing antigens from more than one pathogenic organism within a single dose are known as “multivalent” or “combination” vaccines. Combination vaccines that have been approved for human use in the EU or the USA include: bivalent vaccines against hepatitis A virus (HAV) and hepatitis B virus (HBV) e.g. TIVINRIX™ [1]; bivalent vaccines against type b Haemophilus influenzae (Hib) and HBV e.g. COMVAX™ [2]; trivalent MMR vaccines against measles, mumps and rubella e.g. PRIORIX™ [3]; trivalent vaccines against diphtheria (D), tetanus (T) and pertussis (P) e.g. INFANRIX™ [4]; tetravalent vaccines against DTP-Hib e.g. TRIHIBIT™ [5]; pentavalent vaccines against DTP-HBV-polio e.g. INFANRIX-PENTA™ [6] and PEDIARIX™; and hexavalent vaccines against DTP-HBV-polio-Hib e.g. INFANRIX-HEXA™ [7] and HEXAVAC™ [8].

Combination vaccines offer patients the advantage of receiving a reduced number of injections, which leads to the clinical advantage of increased compliance for pediatric vaccination. At the same time, however, they can present manufacturing difficulties due to factors including: physical and biochemical incompatibility between antigens; immunological interference; and stability.

The inclusion of non-antigen components in vaccines is necessary but controversial. Such components include preservatives. These are used to prevent bacterial growth in vaccines, because the cloudy nature of adsorbed vaccine preparations means that bacterial growth can evade visual detection. This issue is particularly important when vaccines are packaged in multidose containers.

One such preservative is 1-hydroxy-2-phenoxyethane (also known as ‘2-hydroxyethyl phenyl ether’, ‘2-phenoxyethanol’, ‘ethyleneglycol phenyl ether’, etc.; see ref. 9), which is a component of vaccines such as HAVRIX™, DAPTACEL™, IPOL™, TETRAVAC™, PEDIARIX™ and the various INFANRIX™ products. It has the formula C₈H₁₀O₂ and CAS number 122-99-6:

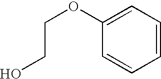

Although the safety profile of 1-hydroxy-2-phenoxyethane is better than that of mercurial preservatives (e.g. thiomersal), it is the weaker antimicrobial of the two [10] and so should be included at higher levels. On the other hand, it has been suggested that 1-hydroxy-2-phenoxyethane in vaccines may be linked to the development of eczema in patients [11], and it has been said to be a neurotoxin [12], so some patient groups are against its use in vaccines [13].

When adding further antigens to existing combination vaccines, however, it may not be feasible (e.g. for historical or regulatory reasons) to avoid this preservative. It is an object of the invention to provide methods for manufacturing combination vaccines that contain 1-hydroxy-2-phenoxyethane, wherein the use of 1-hydroxy-2-phenoxyethane is controlled such that its levels can kept low in the final vaccine.

### DETAILED DESCRIPTION OF THE INVENTION

The invention provides processes for preparing combination vaccines that include diphtheria and tetanus toxoids. These two components are used in the processes as a single component containing both toxoids, and also containing 1-hydroxy-2-phenoxyethane. Thus the invention provides a process for manufacturing a combination vaccine, wherein:
(a) the vaccine comprises a diphtheria toxoid, a tetanus toxoid, and at least one further antigen;
(b) the process involves mixing a first component and a second component, wherein the first component comprises a diphtheria toxoid, a tetanus toxoid and 1-hydroxy-2-phenoxyethane, and the second component comprises said at least one further antigen.

In this process, the second component may include one or more of (i) a hepatitis B surface antigen, (ii) one or more poliovirus antigens, (iii) one or more acellular pertussis antigens, (iv) a conjugate of the capsular saccharide antigen from Haemophilus influenzae type B, (v) a conjugate of the capsular saccharide antigen from serogroup C of Neisseria meningitidis, (vi) a conjugate of the capsular saccharide antigen from serogroup Y of Neisseria meningitidis, (vii) a conjugate of the capsular saccharide antigen from serogroup W135 of Neisseria meningitidis, and/or (viii) a conjugate of the capsular saccharide antigen from serogroup A of Neisseria meningitidis. The second component is substantially free from any diphtheria toxoid or tetanus toxoid (except that, in some embodiments, these toxoids or their derivatives may be present as the carrier protein in a conjugate). The second component may include 1-hydroxy-2-phenoxyethane or may be substantially free of 1-hydroxy-2-phenoxyethane. If the second component is substantially free of 1-hydroxy-2-phenoxyethane then the process can include a further step of mixing the first and second components with a source of 1-hydroxy-2-phenoxyethane.

Thus the invention provides a process for manufacturing a combination vaccine, wherein:
(a) the vaccine comprises a diphtheria toxoid, a tetanus toxoid, and at least one further antigen;

and either
(b1) the process involves mixing a first component and a second component, wherein the first component comprises a diphtheria toxoid, a tetanus toxoid and 1-hydroxy-2-phenoxyethane, and the second component comprises said at least one further antigen and 1-hydroxy-2-phenoxyethane;

or
(b2) the process involves mixing a first component, a second component and a third component, wherein the first component comprises a diphtheria toxoid, a tetanus toxoid and 1-hydroxy-2-phenoxyethane, the second component comprises said at least one further antigen but is substantially free of and 1-hydroxy-2-phenoxyethane, and the third component comprises 1-hydroxy-2-phenoxyethane.

Where the process uses step (b2), the third component may be substantially free from antigen, or may include one or more additional further antigen(s).

Combination vaccines are typically manufactured by mixing individual antigenic components that have been separately prepared for use in a number of different vaccines e.g. a hepatitis B surface antigen may be prepared in bulk for use as a monovalent vaccine, as part of a bivalent vaccine, as part of a tetravalent vaccine, etc. An individual antigenic component of a vaccine may thus be used in a number of different ‘modular’ vaccines, and it must be suitable for use in all them (as well as often being a stand-alone vaccine itself). In this modular situation then the simplest way to avoid contamination is to include a preservative in each individual component. According to the invention, however, this simple approach can lead to a final concentration of preservative that may be higher than necessary, and so the invention provides methods where the preservative is added with only a subset of the antigenic components.

Accordingly, a combination vaccine comprising a diphtheria toxoid (‘D’), a tetanus toxoid (‘T’), at least one acellular pertussis antigen (‘aP’), a hepatitis B surface antigen (‘HBsAg’) and poliovirus antigens (‘IPV’) is manufactured by a process in which: (i) the D & T antigens are used in a mixed form that includes a 1-hydroxy-2-phenoxyethane preservative; and (ii) at least one of the aP, HBsAg and IPV antigens is provided without a 1-hydroxy-2-phenoxyethane preservative.

Thus the invention provides a process for manufacturing a combination vaccine, wherein:
(a) the vaccine comprises a diphtheria toxoid, a tetanus toxoid, an acellular pertussis antigen, a hepatitis B surface antigen and poliovirus antigens;
(b) the process comprises the steps of mixing:
   (i) a first antigenic component that comprises both a diphtheria toxoid and a tetanus toxoid,
   (ii) a second antigenic component that comprises a hepatitis B surface antigen,
   (iii) a third antigenic component that comprises one or more poliovirus antigens, and
   (iv) a fourth antigenic component that comprises one or more acellular pertussis antigens,
(c) the first antigenic component also comprises 1-hydroxy-2-phenoxyethane; and
(d) at least one (i.e. 1, 2 or 3) of the second, third and fourth antigenic components is substantially free from 1-hydroxy-2-phenoxyethane.

In this process the four components (i) to (iv) can be mixed with each other in any order.

Preferably, none of the HBsAg, IPV and aP components includes 1-hydroxy-2-phenoxyethane, but additional preservative is added separately. Thus the invention provides a process for manufacturing a combination vaccine, wherein:
(a) the vaccine comprises a diphtheria toxoid, a tetanus toxoid, an acellular pertussis antigen, a hepatitis B surface antigen and poliovirus antigens;
(b) the process comprises the steps of mixing:
   (i) a first antigenic component that comprises both a diphtheria toxoid and a tetanus toxoid,
   (ii) a second antigenic component that comprises a hepatitis B surface antigen,
   (iii) a third antigenic component that comprises one or more poliovirus antigens,
   (iv) a fourth antigenic component that comprises one or more acellular pertussis antigens, and
   (v) a preservative component that comprises 1-hydroxy-2-phenoxyethane;
(c) the first antigenic component also comprises 1-hydroxy-2-phenoxyethane;
(d) each of the second, third and fourth antigenic components is substantially free from 1-hydroxy-2-phenoxyethane; and
(e) the preservative component is substantially free from any diphtheria toxoid, tetanus toxoid, acellular pertussis antigens, hepatitis B surface antigen and poliovirus antigens.

In this process the five components (i) to (v) can be mixed with each other in any order.

By using these processes, the level of 1-hydroxy-2-phenoxyethane in the final combination vaccine can be controlled simply by controlling (1) the level of 1-hydroxy-2-phenoxyethane in the combined diphtheria/tetanus first antigenic component, (2) the degree by which that antigenic component is diluted during manufacture, and (3) the amount of any 1-hydroxy-2-phenoxyethane added in the preservative component.

For all these processes, the final vaccine that is prepared preferably includes about 5 mg/ml of 1-hydroxy-2-phenoxyethane.

The processes of the invention will generally involve the further step of packaging the combination vaccine in a unit dose form e.g. into vials, pre-filled syringes, etc.

Processes of the invention are particularly suitable for manufacturing a pentavalent (for protecting against five pathogenic organisms) combination vaccine in which: (a) antigens are present at the following concentrations per milliliter (±10%): 50 Lf diphtheria toxoid; 20 Lf tetanus toxoid; 50 μg inactivated pertussis toxin; 50 μg filamentous hemagglutinin; 16 μg pertactin; 20 μg HBsAg; 80 DU Type 1 poliovirus; 16 DU Type 2 poliovirus; and 64 DU Type 3 poliovirus, and (b) 1-hydroxy-2-phenoxyethane is present at about 5 mg/ml. They are also suitable for manufacturing 6-valent, 7-valent, 8-valent, 9-valent, etc., vaccines.

### The Mixture of Diphtheria and Tetanus Antigens

Processes of the invention involves mixing various antigenic components. One of these is a mixture that includes both a diphtheria toxoid and a tetanus toxoid. Prior to being used in the process of the invention, therefore, a diphtheria toxoid and a tetanus toxoid have been mixed, to give a mixture suitable for use as a first antigenic component in a process of the invention.

Diphtheria is caused by Corynebacterium diphtheriae, a Gram-positive non-sporing aerobic bacterium. This organism expresses a prophage-encoded ADP-ribosylating exotoxin (‘diphtheria toxin’), which can be treated (e.g. using formaldehyde) to give a toxoid that is no longer toxic but that remains antigenic and is able to stimulate the production of specific anti-toxin antibodies after injection. Diphtheria toxoids are disclosed in more detail in chapter 13 of reference 14. Preferred diphtheria toxoids are those prepared by formaldehyde treatment. The diphtheria toxoid can be obtained by growing C. diphtheriae in growth medium (e.g. Fenton medium, or Linggoud & Fenton medium), which may be supplemented with bovine extract, followed by formaldehyde treatment, ultrafiltration and precipitation. The toxoided material may then be treated by a process comprising sterile filtration and/or dialysis.

Tetanus is caused by Clostridium tetani, a Gram-positive, spore-forming bacillus. This organism expresses an endopeptidase (‘tetanus toxin’), which can be treated to give a toxoid that is no longer toxic but that remains antigenic and is able to stimulate the production of specific anti-toxin antibodies after injection. Tetanus toxoids are disclosed in more detail in chapter 27 of reference 14. Preferred tetanus toxoids are those prepared by formaldehyde treatment. The tetanus toxoid can be obtained by growing C. tetani in growth medium (e.g. a Latham medium derived from bovine casein), followed by formaldehyde treatment, ultrafiltration and precipitation. The material may then be treated by a process comprising sterile filtration and/or dialysis.

Quantities of diphtheria and tetanus toxoids can be expressed in international units (IU). For example, the NIBSC [15] supplies the ‘Diphtheria Toxoid Adsorbed Third International Standard 1999’ [16,17], which contains 160 IU per ampoule, and the ‘Tetanus Toxoid Adsorbed Third International Standard 2000’ [18,19], which contains 469 IU per ampoule. As an alternative to the IU system, the ‘Lf’ unit (“flocculating units”, the “limes flocculating dose”, or the “limit of flocculation”) is defined as the amount of toxoid which, when mixed with one International Unit of antitoxin, produces an optimally flocculating mixture [20]. For example, the NIBSC supplies ‘Diphtheria Toxoid, Plain’ [21], which contains 300 Lf per ampoule, ‘The 1st International Reference Reagent For Diphtheria Toxoid For Flocculation Test’ [22] which contains 900 Lf per ampoule, and ‘The 1st International Reference Reagent for Tetanus Toxoid For Flocculation Test’ [23] which contains 1000 LF per ampoule. The conversion between IU and Lf systems depends on the particular toxoid preparation.

The diphtheria and tetanus toxoids in the first component are preferably adsorbed (more preferably totally adsorbed) onto an aluminum hydroxide adjuvant. This can be achieved by preparing the toxoids separately, adsorbing each of them separately to an aluminum hydroxide adjuvant, and then mixing the two adsorbed toxoids (optionally with further adjuvant) to give the material for use in the processes of the invention.

The ratio (measured in Lf units) of diphtheria toxoid to tetanus toxoid in the first component is usually between 2:1 and 3:1, and is preferably about 2.5:1.

The first component also includes 1-hydroxy-2-phenoxyethane. The amount of 1-hydroxy-2-phenoxyethane in the first component is preferably (a) between 2.5 mg and 3.5 mg (e.g. about 3 mg) for every 100 Lf of diphtheria toxoid, and/or (b) between 7 mg and 8 mg (e.g. about 7.5 mg) for every 100 Lf of tetanus toxoid. A 1-hydroxy-2-phenoxyethane concentration of between 3 g/l and 8 g/l (e.g. between 4-6 g/l, or about 5 g/l) in the first component is preferred.

The first component will generally be in aqueous form, and the concentrations of sub-components are preferably as follows, expressed per milliliter (±10%): 167 Lf diphtheria toxoid; 67 Lf tetanus toxoid; 5 mg 1-hydroxy-2-phenoxyethane. As an alternative, the concentrations of sub-components may be fractions of these values, provided that the relative proportions (the ratio) stay the same e.g. as obtainable by simple dilution.

The first antigenic component may include further compounds in addition to the toxoids, adjuvant and preservative. In particular, it may comprise residual formaldehyde and/or sodium chloride. A sodium chloride concentration of between 8 and 9 mg/ml (e.g. about 8.5 mg/ml) is preferred.

Where bovine materials are used in the culture of C. tetani and/or C. diphtheriae, they should be obtained from sources that are free from bovine spongiform encephalopathy (BSE) or from other transmissible spongiform encephalopathies (TSEs).

The first antigenic component is substantially free from polysorbate 80 e.g. it contains less than 0.1 μg/ml of polysorbate 80, and preferably contains no detectable polysorbate 80.

The first antigenic component is substantially free from mercurial preservatives (e.g. thimerosal) e.g. it contains less than 0.1 μg/ml of mercury, and preferably contains no detectable mercury.

### The Hepatitis B Surface Antigen

The process of the invention involves mixing various antigenic components. One of these components can comprise a hepatitis B surface antigen (HBsAg).

Hepatitis B virus (HBV) is one of the known agents which causes viral hepatitis. The HBV virion consists of an inner core surrounded by an outer protein coat or capsid. The viral core contains the viral DNA genome. The major component of the capsid is a protein known as HBV surface antigen or, more commonly, ‘HBsAg’. All existing hepatitis B vaccines contain HBsAg, and when this antigen is administered to a vaccinee it stimulates the production of anti-HBsAg antibodies which protect against HBV infection.

For vaccine manufacture, HBsAg can be made in two ways. The first method involves purifying the antigen in particulate form from the plasma of chronic hepatitis B carriers, as large quantities of HBsAg are synthesized in the liver and released into the blood stream during an HBV infection. The second way involves expressing the protein by recombinant DNA methods. HBsAg for use with the method of the invention may be prepared in either way, but it is preferred to use HBsAg which has been recombinantly expressed e.g. in a yeast, such as a Saccharomyces e.g. in S. cerevisiae. Unlike native HBsAg (i.e. as in the plasma-purified product), yeast-expressed HBsAg is generally non-glycosylated, and this is the most preferred form of HBsAg for use with the invention.

Yeast-expressed HBsAg is advantageously in the form of substantially-spherical particles (average diameter of about 20 nm), including a lipid matrix comprising phospholipids. Unlike plasma-derived HBsAg particles, yeast-expressed particles may include phosphatidylinositol. Moreover, the lipid matrix may include a non-ionic surfactant, such as polysorbate 20, which may be incorporated into the matrix during purification of the antigen from a yeast expression host. Using polysorbate 20 during disruption of recombinant yeast cells at the start of HBsAg purification is one way in which it can be introduced into the HBsAg particles. Any polysorbate 20 will typically be present at a weight ratio of at least 5 μg per 100 μg of HBsAg e.g. up to 50 μg per 100 μg of HbsAg.

All known HBV subtypes contain the common determinant ‘a’. Combined with other determinants and subdeterminants, nine subtypes have been identified: ayw1, ayw2, ayw3, ayw4, ayr, adw2, adw4, adrq− and adrq+. Besides these subtypes, other variants have emerged, such as HBV mutants that have been detected in immunised individuals (“escape mutants”). The most preferred HBV subtype for use with the invention is subtype adw2. A preferred HBsAg has the following amino acid sequence (SEQ ID NO: 1):

MENITSGFLGPLLVLQAGFFLLTRILTIPQSLDSWWTSLNFLGGSPVCLG
QNSQSPTSNHSPTSCPPICPGYRWMCLRRFIIFLFILLLCLIFLLVLLDY
QGMLPVCPLIPGSTTTNTGPCKTCTTPAQGNSMFPSCCCTKPTDGNCTCI
PIPSSWAFAKYLWEWASVRFSWLSLLVPFVQWFVGLSPTVWLSAIWMMWY
WGPSLYSIVSPFIPLLPIFFCLWVYI

This sequence differs from the closest database matches at amino acid 117, having an Asn residue rather than Ser. The invention can use SEQ ID NO: 1, or a sequence differing from SEQ ID NO: 1 by up to 10 (i.e. 1, 2, 3, 4, 5, 6, 7, 8, 9 or 10) single amino acid substitutions.

In addition to the ‘S’ sequence, a surface antigen may include all or part of a pre-S sequence, such as all or part of a pre-S1 and/or pre-S2 sequence.

Various options are available for yeast-based expression, including choices of yeast strain, culture conditions, promoter for control of recombinant expression, gene termination sequences, codon usage, etc. According to the invention, the HBsAg is preferably expressed: (1) under the control of an upstream promoter from a glyceraldehyde-3-phosphate dehydrogenase gene; and/or (2) with a downstream ARG3 transcription terminator.

Glyceraldehyde-3-phosphate dehydrogenase is a glycolytic enzyme, and its promoter has been found to be particularly suitable for controlling expression of HBsAg in S. cerevisiae [24]. A preferred GAPDH promoter comprises the following 1060-mer nucleotide sequence (SEQ ID NO: 2):

AAGCTTACCAGTTCTCACACGGAACACCACTAATGGACACACATTCGAAA
TACTTTGACCCTATTTTCGAGGACCTTGTCACCTTGAGCCCAAGAGAGCC
AAGATTTAAATTTTCCTATGACTTGATGCAAATTCCCAAAGCTAATAACA
TGCAAGACACGTACGGTCAAGAAGACATATTTGACCTCTTAACAGGTTCA
GACGCGACTGCCTCATCAGTAAGACCCGTTGAAAAGAACTTACCTGAAAA
AAACGAATATATACTAGCGTTGAATGTTAGCGTCAACAACAAGAAGTTTA
CTGACGCGGAGGCCAAGGCAAAAAGATTCCTTGATTACGTAAGGGAGTTA
GAATCATTTTGAATAAAAAACACGCTTTTTCAGTTCGAGTTTATCATTAT
CAATACTGCCATTTCAAAGAATACGTAAATAATTAATAGTAGTGATTTTC
CTAACTTTATTTAGTCAAAAAATTAGCCTTTTAATTCTGCTGTAACCCGT
ACATGCCCAAAATAGGGGGCGGGTTACACAGAATATATAACATCGTAGGT
GTCTGGGTGAACAGTTTATTCCTGGCATCCACTAAATATAATGGAGCCCG
CTTTTTAAGCTGGCATCCAGAAAAAAAAAGAATCCCAGCACCAAAATATT
GTTTTCTTCACCAACCATCAGTTCATAGGTCCATTCTCTTAGCGCAACTA
CAGAGAACAGGGGCACAAACAGGCAAAAAACGGGCACAACCTCAATGGAG
TGATGCAACCTGCCTGGAGTAAATGATGACACAAGGCAATTGACCCACGC
ATGTATCTATCTCATTTTCTTACACCTTCTATTACCTTCTGCTCTCTCTG
ATTTGGAAAAAGCTGAAAAAAAAGGTTGAAACCAGTTCCCTGAAATTATT
CCCCTACTTGACTAATAAGTATATAAAGACGGTAGGTATTGATTGTAATT
CTGTAAATCTATTTCTTAAACTTCTTAAATTCTACTTTTATAGTTAGTCT
TTTTTTTAGTTTTAAAACACCAAGAACTTAGTTTCGAATAAACACACATA
AACAAACAAA

This sequence differs from the sequence in reference 24 as follows: (1) A/C substitution at nucleotide 42; (2) T/A substitution at nucleotide 194; (3) C/A mutation at nucleotide 301; (4) A insertion at nucleotide 471; (5) C/T substitution at residue 569; (6) T/C substitution at residue 597; (7) T insertion at nucleotide 604 (penta-T instead of tetra-T); and (8) replacement of 3′ GCTT sequence with a single A.

The invention can use this 1060-mer promoter sequence, or a sequence differing from this 1060-mer sequence by up to 20 (i.e. 1, 2, 3, 4, 5, 6, 7, 8, 9, 10, 11, 12, 13, 14, 15, 16, 17, 18, 19 or 20) point mutations, each point mutation being the deletion, substitution or insertion of a single nucleotide.

The 1060-mer sequence is preferably immediately downstream of the ATG start codon encoding the N-terminus of the HBsAg (SEQ ID NO: 3):

AAGCTTACCAGTTCTCACACGGAACACCACTAATGGACACACATTCGAAATACTTTGACCCTATTTTC
GAGGACCTTGTCACCTTGAGCCCAAGAGAGCCAAGATTTAAATTTTCCTATGACTTGATGCAAATTCC
CAAAGCTAATAACATGCAAGACACGTACGGTCAAGAAGACATATTTGACCTCTTAACAGGTTCAGACG
CGACTGCCTCATCAGTAAGACCCGTTGAAAAAGAACTTACCTGAAAAAACGAATATATACTAGCGTTG
AATATTAGCGTCAACAACAAGAAGTTTACTGACGCGGAGGCCAAGGCAAAAAGATTCCTTGATTACGT
AAGGGAGTTAGAATCATTTTGAATAAAAAACACGCTTTTTCAGTTCGAGTTTATCATTATCAATACTG
CCATTTCAAAGAATACGTAAATAATTAATAGTAGTGATTTTCCTAACTTTATTTAGTCAAAAAATTAG
CCTTTTAATTCTGCTGTAACCCGTACATGCCCAAAATAGGGGGCGGGTTACACAGAATATATAACATC
GTAGGTGTCTGGGTGAACAGTTTATTCCTGGCATCCACTAAATATAATGGAGCCCGCTTTTTAAGCTG
GCATCCAGAAAAAAAAAGAATCCCAGCACCAAAATATTGTTTTCTTCACCAACCATCAGTTCATAGGT
CCATTCTCTTAGCGCAACTACAGAGAACAGGGGCACAAACAGGCAAAAAACGGGCACAACCTCAATGG
AGTGATGCAACCTGCCTGGAGTAAATGATGACACAAGGCAATTGACCCACGCATGTATCTATCTCATT
TTCTTACACCTTCTATTACCTTCTGCTCTCTCTGATTTGGAAAAAGCTGAAAAAAAAGGTTGAAACCA
GTTCCCTGAAATTATTCCCCTACTTGACTAATAAGTATATAAAGACGGTAGGTATTGATTGTAATTCT
GTAAATCTATTTCTTAAACTTCTTAAATTCTACTTTTATAGTTAGTCTTTTTTTTAGTTTTAAAACAC
CAAGAACTTAGTTTCGAATAAACACACATAAACAAACAAAATG...

The ARG3 gene in yeast encodes the ornithine carbamoyltransferase enzyme [25] and its transcription termination sequence has been used in several yeast recombinant expression systems [26, 27, 28]. It is advantageous for the control of HBsAg expression in yeast, particularly in combination with a GAPDH promoter.

The gene encoding HBsAg will typically be an insert in a plasmid. A preferred plasmid includes a GAPDH promoter, followed by a sequence encoding HBsAg, followed by an ARG3 terminator. Preferred plasmids may also include one, two or all three of: (1) a LEU2 selection marker; (2) a 2μ plasmid sequence; and/or (3) an origin of replication functional in Escherichia coli [28]. Thus preferred plasmids can act as shuttle vectors between yeast and E. coli.

A plasmid with between 14500 and 15000 by is preferred e.g. between 14600 and 14700 bp.

Where a LEU2 selection marker is used then the host cell should be LEU2^{-ve} (i.e. a leucine auxotroph). The host cell may be a leu2-3 leu2-112 mutant. Further characteristics of preferred yeast hosts are his3 and/or can1-11. A most preferred yeast host is leu2-3 leu2-112 his3 can1-11, such as the DC5 strain.

Yeast may be cultured in a synthetic medium e.g. containing purified amino acids (optionally ensuring that leucine is included), vitamins, salts, etc. HBsAg can then be purified by a process involving steps such as precipitation, ion exchange chromatography, and ultrafiltration. Other steps that may be used during its purification include gel permeation chromatography, and caesium chloride ultracentrifugation.

Although HBsAg may be adsorbed to an aluminum hydroxide adjuvant in the final vaccine (as in the well-known ENGERIX-B™ product), or may remain unadsorbed, it will generally be adsorbed to an aluminum phosphate adjuvant prior to being used in the process of the invention. Details of HBsAg adsorption to this adjuvant can be found in reference 29. Total adsorption is preferred.

After purification (e.g. before adsorption to adjuvant) HBsAg may be subjected to dialysis (e.g. with cysteine), which can be used to remove any mercurial preservatives such as thimerosal that may have been used during HBsAg preparation [30].

Quantities of HBsAg are typically expressed in micrograms.

Preferably, the HBsAg component is substantially free from polysorbate 80 e.g. it contains less than 0.1 μg/ml of polysorbate 80, and preferably contains no detectable polysorbate 80.

The HBsAg component is preferably substantially free from 1-hydroxy-2-phenoxyethane e.g. it contains less than 0.1 μg/ml of 1-hydroxy-2-phenoxyethane, and preferably contains no detectable 1-hydroxy-2-phenoxyethane.

### The Poliovirus Antigen(s)

The process of the invention involves mixing various antigenic components. One of these components can comprise one or more poliovirus antigen(s).

Poliomyelitis can be caused by one of three types of poliovirus. The three types are similar and cause identical symptoms, but they are antigenically very different and infection by one type does not protect against infection by others. As explained in chapter 24 of reference 14, it is therefore preferred to use three poliovirus antigens in the process of the invention—poliovirus Type 1 (e.g. Mahoney strain), poliovirus Type 2 (e.g. MEF-1 strain), and poliovirus Type 3 (e.g. Saukett strain).

Polioviruses may be grown in cell culture. A preferred culture uses a Vero cell line, which is a continuous cell line derived from monkey kidney. Vero cells can conveniently be cultured microcarriers. Culture of the Vero cells before and during viral infection may involve the use of bovine-derived material, such as calf serum, and of lactalbumin hydrolysate (e.g. obtained by enzymatic degradation of lactalbumin). Such bovine-derived material should be obtained from sources which are free from BSE or other TSEs.

After growth, virions may be purified using techniques such as ultrafiltration, diafiltration, and chromatography. Prior to administration to patients, polioviruses must be inactivated, and this can be achieved by treatment with formaldehyde before the viruses are used in the process of the invention.

The viruses are preferably grown, purified and inactivated individually, and are then combined to give a bulk mixture for use in the process of the invention. The combined polioviruses have preferably not been adsorbed to any adjuvant before they are used in the process of the invention, but after the addition they may become adsorbed onto aluminum adjuvant(s) in the vaccine composition.

Quantities of poliovirus are typically expressed in the ‘DU’ unit (the “D-antigen unit” [31]).

Preferably, the poliovirus component is substantially free from polysorbate 80 e.g. it contains less than 0.1 μg/ml of polysorbate 80, and preferably contains no detectable polysorbate 80. Polysorbate 80 may, however, have been used during production of the poliovirus component.

Preferably, the poliovirus component is substantially free from mercurial preservatives (e.g. thimerosal) e.g. it contains less than 0.1 μg/ml of mercury, and preferably contains no detectable mercury.

Preferably, the poliovirus component is substantially free from 1-hydroxy-2-phenoxyethane e.g. it contains less than 0.1 μg/ml of 1-hydroxy-2-phenoxyethane, and preferably contains no detectable 1-hydroxy-2-phenoxyethane.

### The Acellular Pertussis Antigen(s)

The process of the invention involves mixing various antigenic components. One of these components can comprise one or more acellular pertussis antigen(s).

Bordetella pertussis is a Gram-negative non-sporing aerobic bacillus that causes whooping cough. As described in more detail in chapter 21 of reference 14, vaccines against B. pertussis have been available for many years, and fall into two categories: cellular and acellular. Cellular vaccines comprise whole B. pertussis cells which have been killed and deactivated (e.g. by treatment with formalin), whereas acellular vaccines comprise specific purified B. pertussis antigens, either purified from the native bacterium or purified after expression in a recombinant host.

The present invention utilizes acellular pertussis antigens. In particular, the second antigenic component that is used in the process of the invention includes one, two or three of the following well-known and well-characterized B. pertussis antigens: (1) detoxified pertussis toxin (pertussis toxoid, or ‘PT’); (2) filamentous hemagglutinin (‘FHA’); (3) pertactin (also known as the ‘69 kiloDalton outer membrane protein’). It is most preferred that all three of these antigens should be included in the vaccine of the invention, and this may best be achieved by having all three antigens in the second antigenic component (i.e. they are mixed before the process of the invention is performed), although it is also possible to add at least one of them separately (i.e. the second antigenic component used in the process of the invention includes one or two acellular antigens, with the other(s) being added separately e.g. as a fifth (and possibly a sixth) antigenic component).

These three antigens are preferably prepared by isolation from B. pertussis culture grown in modified Stainer-Scholte liquid medium. PT and FHA can be isolated from the fermentation broth (e.g. by adsorption on hydroxyapatite gel), whereas pertactin can be extracted from the cells by heat treatment and flocculation (e.g. using barium chloride).

The antigens can be purified in successive chromatographic and/or precipitation steps. PT and FHA can be purified by hydrophobic chromatography, affinity chromatography and size exclusion chromatography. Pertactin can be purified by ion exchange chromatography, hydrophobic chromatography and size exclusion chromatography.

FHA and pertactin may be treated with formaldehyde prior to use according to the invention. PT is preferably detoxified by treatment with formaldehyde and/or glutaraldehyde. As an alternative to this chemical detoxification procedure the PT may be a mutant PT in which enzymatic activity has been reduced by mutagenesis [32], but detoxification by chemical treatment is preferred.

The pertussis antigen(s) in the second antigenic component used in the process of the invention are preferably adsorbed onto one or more aluminum salt adjuvants. As an alternative, they may be added in an unadsorbed state. Where pertactin is added then it is preferably adsorbed onto an aluminum hydroxide adjuvant before being used in the process of the invention. PT and FHA may be adsorbed onto an aluminum hydroxide adjuvant or an aluminum phosphate before being used in the process of the invention. Adsorption of all of PT, FHA and pertactin to aluminum hydroxide is most preferred.

Further acellular pertussis antigens that can be used include fimbriae (e.g. agglutinogens 2 and 3).

Quantities of acellular pertussis antigens are typically expressed in micrograms.

Preferably, the acellular pertussis antigen component is substantially free from 1-hydroxy-2-phenoxyethane e.g. it contains less than 0.1 μg/ml of 1-hydroxy-2-phenoxyethane, and preferably contains no detectable 1-hydroxy-2-phenoxyethane.

Preferably, the acellular pertussis component is substantially free from mercurial preservatives (e.g. thimerosal) e.g. it contains less than 0.1 μg/ml of mercury, and preferably contains no detectable mercury.

The acellular pertussis component may include polysorbate 80. The level of polysorbate 80 in the pertussis component is such that, after any dilution of the component to ensure that the pertussis antigens are at the concentration desired in the final vaccine product, the polysorbate 80 concentration is less than 200 μg/ml, and is preferably less than 100 μg/ml. A typical polysorbate concentration in the final vaccine product is between 70 and 90 μg/ml e.g. about 80 μg/ml.

A preferred upper limit on the amount of polysorbate 80 in the pertussis component prior to its use in the process of the invention is <40 μg per 10 μg of FHA.

### Further Antigens

As well as including D, T, Pa, HBsAg and poliovirus antigens, vaccines prepared by the process of the invention may include antigens from further pathogens, and thus the process may involve a step in which a further antigenic component is added during mixing, wherein the further antigenic component does not comprise any of D, T, Pa, HBsAg and poliovirus antigens. The antigens in such further antigenic components may be derived from a pathogen selected from the group consisting of: Neisseria meningitidis (one or more of serogroups A, B, C, W135 and/or Y); Streptococcus pneumoniae; Haemophilus influenzae; Moraxella catarrhalis; hepatitis A virus; measles virus; mumps virus; rubella virus; rotavirus; and influenza virus. These further antigens may or may not be adsorbed to an aluminum salt adjuvant in the final vaccine.

Preferred antigens to be used during processes of the invention are: (1) a capsular saccharide from Haemophilus influenzae type B conjugated to a carrier protein; (2) a capsular saccharide from N. meningitidis serogroup C, conjugated to a carrier protein; (3) a capsular saccharide from N. meningitidis serogroup Y, conjugated to a carrier protein; and/or (4) a capsular saccharide from a S. pneumoniae, conjugated to a carrier protein. Combinations of (1)+(2) or of (1)+(2)+(3) are useful.

Various proteins are known for use as carriers, and preferred carrier proteins are bacterial toxins or toxoids, such as diphtheria toxoid or tetanus toxoid. Other suitable carrier proteins include, but are not limited to, the CRM197 mutant of diphtheria toxin [33-35], the N. meningitidis outer membrane protein [36], synthetic peptides [37, 38], heat shock proteins [39,40], pertussis proteins [41,42], cytokines [43], lymphokines [43], hormones [43], growth factors [43], artificial proteins comprising multiple human CD4⁺ T cell epitopes from various pathogen-derived antigens [44] such as N19 [45], protein D from H. influenzae [46,47], pneumococcal surface protein PspA [48], pneumolysin [49], iron-uptake proteins [50], toxin A or B from C. difficile [51], S. agalactiae proteins [52], etc.

The carrier protein used for a H. influenzae conjugate is preferably a tetanus toxoid (to give the ‘PRP-T’ product). The weight ratio of saccharide to carrier in the PRP-T is preferably between 1:2 and 1:4 e.g. between 1:2.5 and 1:3.5. A weight ratio between 2.8:1 and 3.2:1 can be used, and a weight ratio of about 3:1 is preferred. At a typical dose of 10 μg (measured as saccharide), therefore, a composition of the invention will include 30 μg of tetanus toxoid carrier from PRP-T, plus further tetanus toxoid as the ‘T’ antigen, and also from any other conjugates using a tetanus toxoid carrier. Another preferred conjugate has a weight ratio of about 2.5:1. The mass of Hib saccharide in a vaccine of the invention will usually be in the range of 0.5 μg to 50 μg e.g. from 1-20 μg, from 10-15 μg, from 12-16 μg, etc. The amount may be about 12.5 μg. A mass of less than 5 μg may be suitable [53] e.g. in the range 1-5 μg, 2-4 μg, or about 2.5 μg.

The carrier protein used for a N. meningitidis conjugate may also be a tetanus toxoid. The weight ratio of saccharide to carrier in such a meningococcal conjugate is preferably between 0.5:1 and 1:0.5 e.g. about 1:1.

These conjugates can be made by various processes. For example, one process involves activating a Hib capsular polysaccharide using cyanogen bromide, coupling the activated saccharide to an adipic acid linker (such as (1-ethyl-3-(3-dimethylaminopropyl)carbodiimide), typically the hydrochloride salt), and then reacting the linker-saccharide entity with a tetanus toxoid carrier protein. Carbodiimide condenation can also be used for conjugate production [54].

Attachment of a saccharide to a carrier is preferably via a —NH₂ group e.g. in the side chain of a lysine residue in a carrier protein, or of an arginine residue. Where a saccharide has a free aldehyde group then this can react with an amine in the carrier to form a conjugate by reductive amination.

As described in reference 55, a mixture can include one conjugate with direct saccharide/protein linkage and another conjugate with linkage via a linker. This arrangement applies particularly when using saccharide conjugates from different meningococcal serogroups e.g. MenA and MenC saccharides may be conjugated via a linker, whereas MenW135 and MenY saccharides may be conjugated directly to a carrier protein.

In general, conjugates with a saccharide:protein ratio (w/w) of between 1:5 (i.e. excess protein) and 5:1 (i.e. excess saccharide) are preferred e.g. ratios between 1:2 and 5:1 and ratios between 1:1.25 and 1:2.5. As described in reference 56, if different meningococcal serogroup conjugates are included in a mixture then these can have different saccharide:protein ratios e.g. one may have a ratio of between 1:2 & 1:5, whereas another has a ratio between 5:1 & 1:1.99.

It is possible to include more than one type of carrier protein in a composition e.g. to reduce the risk of carrier suppression.

Compositions may include a small amount of free carrier. When a given carrier protein is present in both free and conjugated form in a composition of the invention, the unconjugated form is preferably no more than 5% of the total amount of the carrier protein in the composition as a whole, and more preferably present at less than 2% by weight.

As an alternative to adding Hib and/or meningococcal conjugate antigen(s) during manufacture, they may be added to a combination vaccine at the point of delivery. In such a situation, the conjugated antigen is thus contained separately from the other antigens at the point of manufacture, ready for reconstitution at the point of use. The conjugate antigen is preferably packaged within the same package as other antigens.

When contained separately, conjugates will typically be freeze-dried (lyophilized) in a separate container, such that the packaged vaccine will contain at least two separate containers. Prior to administration to a patient, the freeze-dried material will be reconstituted and diluted with the liquid from the other container. Typically, therefore, the conjugate container will be a vial and the other container will contain a liquid within a vial or a pre-filled syringe. The liquid contents of the second container will be transferred into the vial containing the freeze-dried antigen(s), thereby reconstituting them for administration to a patient.

The conjugate container is preferably a vial which has a cap (e.g. a Luer lock) adapted such that a pre-filled syringe can be inserted into the cap, the contents of the syringe can be expelled into the vial to reconstitute the freeze-dried material therein, and the contents of the vial can be removed back into the syringe. After removal of the syringe from the vial, a needle can then be attached and the vaccine can be administered to a patient. The cap is preferably located inside a seal or cover, such that the seal or cover has to be removed before the cap can be accessed.

Thus the invention provides a process for preparing a two-container combination vaccine, comprising the following steps:
preparing a combination vaccine as described above, but wherein the said one or more antigens does not include a H. influenzae type B antigen;
packaging said combination vaccine in a first container (e.g. a syringe);
preparing a freeze-dried H. influenzae type B antigen;
packaging said freeze-dried H. influenzae type B antigen in a second container (e.g. a vial); and
packaging the first container and second container together in a kit.
   The kit can then be distributed to physicians. The kit may also include one or more further container(s) that include antigen(s) for immunisation e.g. pneumococcal conjugates.

The second container (i.e. the container in which the lyophilized Hib antigen is present) may also contain a conjugated N. meningitidis serogroup C saccharide antigen (‘MenC’) and/or a conjugated N. meningitidis serogroup Y saccharide antigen (‘MenY’). Such conjugates can be prepared using the methods described in, for example, references 57, 58 and 59.

Where a component includes a meningococcal saccharide conjugate, there may be one or more than one such conjugate. Including 2, 3, or 4 of serogroups A, C, W135 and Y is typical e.g. A+C, A+W135, A+Y, C+W135, C+Y, W135+Y, A+C+W135, A+C+Y, A+W135+Y, A+C+W135+Y, etc. Components including saccharides from all four of serogroups A, C, W135 and Y are useful, as in the MENACTRA™ product. Where conjugates from more than one serogroup are included then they may be present at substantially equal masses e.g. the mass of each serogroup's saccharide is within ±10% of each other. A typical quantity per serogroup in the lyophilised component is between 1 μg and 20 μg e.g. between 2 and 10 μg per serogroup, or about 4 μg or about 5 μg or about 10 μg. As an alternative to a substantially equal ratio, a double mass of serogroup A saccharide may be used.

The capsular saccharide of serogroup A meningococcus is a homopolymer of (α1→6)-linked N-acetyl-D-mannosamine-1-phosphate, with partial O-acetylation in the C3 and C4 positions. Acetylation at the C-3 position can be 70-95%. Conditions used to purify the saccharide can result in de-O-acetylation (e.g. under basic conditions), but it is useful to retain OAc at this C-3 position. In some embodiments, at least 50% (e.g. at least 60%, 70%, 80%, 90%, 95% or more) of the mannosamine residues in a serogroup A saccharides are O-acetylated at the C-3 position. Acetyl groups can be replaced with blocking groups to prevent hydrolysis [60], and such modified saccharides are still serogroup A saccharides within the meaning of the invention.

The serogroup C capsular saccharide is a homopolymer of (α 2→9)-linked sialic acid (N-acetyl neuraminic acid, or ‘NeuNAc’). The saccharide structure is written as →9)-Neu p NAc 7/8 OAc− (α2→. Most serogroup C strains have O-acetyl groups at C-7 and/or C-8 of the sialic acid residues, but about 15% of clinical isolates lack these O-acetyl groups [61,62]. The presence or absence of OAc groups generates unique epitopes, and the specificity of antibody binding to the saccharide may affect its bactericidal activity against O-acetylated (OAc−) and de-O-acetylated (OAc+) strains [63-65]. Serogroup C saccharides used with the invention may be prepared from either OAc+ or OAc− strains. Licensed MenC conjugate vaccines include both OAc− (NEISVAC-C™) and OAc+ (MENJUGATE™ & MENINGITEC™) saccharides. In some embodiments, strains for production of serogroup C conjugates are OAc+ strains, e.g. of serotype 16, serosubtype P1.7a,1, etc. Thus C:16:P1.7a,1 OAc+ strains may be used. OAc+ strains in serosubtype P1.1 are also useful, such as the C11 strain. Preferred MenC saccharides are taken from OAc+ strains, such as strain C11.

The serogroup W135 saccharide is a polymer of sialic acid-galactose disaccharide units. Like the serogroup C saccharide, it has variable O-acetylation, but at sialic acid 7 and 9 positions [66]. The structure is written as: →4)-D-Neup5Ac(7/9OAc)-α-(2→6)-D-Gal-α-(1→.

The serogroup Y saccharide is similar to the serogroup W135 saccharide, except that the disaccharide repeating unit includes glucose instead of galactose. Like serogroup W135, it has variable O-acetylation at sialic acid 7 and 9 positions [66]. The serogroup Y structure is written as: →4)-D-Neup5Ac(7/9OAc)-α-(2→6)-D-Glc-α-(1→.

The saccharides used according to the invention may be O-acetylated as described above (e.g. with the same O-acetylation pattern as seen in native capsular saccharides), or they may be partially or totally de-O-acetylated at one or more positions of the saccharide rings, or they may be hyper-O-acetylated relative to the native capsular saccharides. For example, reference 67 reports the use of serogroup Y saccharides that are more than 80% de-O-acetylated.

The saccharide moieties in meningococcal conjugates may comprise full-length saccharides as prepared from meningococci, and/or may comprise fragments of full-length saccharides i.e. the saccharides may be shorter than the native capsular saccharides seen in bacteria. The saccharides may thus be depolymerised, with depolymerisation occurring during or after saccharide purification but before conjugation. Depolymerisation reduces the chain length of the saccharides. One depolymerisation method involves the use of hydrogen peroxide [68]. Hydrogen peroxide is added to a saccharide (e.g. to give a final H₂O₂ concentration of 1%), and the mixture is then incubated (e.g. at about 55° C.) until a desired chain length reduction has been achieved. Another depolymerisation method involves acid hydrolysis [69]. Other depolymerisation methods are known in the art. The saccharides used to prepare conjugates for use according to the invention may be obtainable by any of these depolymerisation methods. Depolymerisation can be used in order to provide an optimum chain length for immunogenicity and/or to reduce chain length for physical manageability of the saccharides. In some embodiments, saccharides have the following range of average degrees of polymerisation (Dp): A=10-20; C=12-22; W135=15-25; Y=15-25. In terms of molecular weight, rather than Dp, useful ranges are, for all serogroups: <100 kDa; 5 kDa-75 kDa; 7 kDa-50 kDa; 8 kDa-35 kDa; 12 kDa-25 kDa; 15 kDa-22 kDa. In other embodiments, the average molecular weight for saccharides from each of meningococcal serogroups A, C, W135 and Y may be more than 50 kDa e.g. ≧75 kDa, ≧100 kDa, ≧110 kDa, ≧120 kDa, ≧130 kDa, etc. [70], and even up to 1500 kDa, in particular as determined by MALLS. For instance: a MenA saccharide may be in the range 50-500 kDa e.g. 60-80 kDa; a MenC saccharide may be in the range 100-210 kDa; a MenW135 saccharide may be in the range 60-190 kDa e.g. 120-140 kDa; and/or a MenY saccharide may be in the range 60-190 kDa e.g. 150-160 kDa.

If a component or composition includes both Hib and meningococcal conjugates then, in some embodiments, the mass of Hib saccharide can be substantially the same as the mass of a particular meningococcal serogroup saccharide. In some embodiments, the mass of Hib saccharide will be more than (e.g. at least 1.5×) the mass of a particular meningococcal serogroup saccharide. In some embodiments, the mass of Hib saccharide will be less than (e.g. at least 1.5×) the mass of a particular meningococcal serogroup saccharide.

Where a composition includes saccharide from more than one meningococcal serogroup, there is an mean saccharide mass per serogroup. If substantially equal masses of each serogroup are used then the mean mass will be the same as each individual mass; where non-equal masses are used then the mean will differ e.g. with a 10:5:5:5 μg amount for a MenACWY mixture, the mean mass is 6.25 μg per serogroup. In some embodiments, the mass of Hib saccharide will be substantially the same as the mean mass of meningococcal saccharide per serogroup. In some embodiments, the mass of Hib saccharide will be more than (e.g. at least 1.5×) the mean mass of meningococcal saccharide per serogroup. In some embodiments, the mass of Hib saccharide will be less than (e.g. at least 1.5×) the mean mass of meningococcal saccharide per serogroup [71].

The H. influenzae antigen may be adsorbed onto an aluminum adjuvant prior to being freeze-dried, or it may be free from any aluminum-based adjuvant prior to being freeze-dried. If adsorption to an aluminum salt is used then it is preferred to use an aluminum phosphate rather than a hydroxide. Where multiple conjugates are lyophilized together (e.g. Hib+MenC, or Hib+MenC+MenY) then it is preferred not to include an aluminum salt (or any other adjuvant) in the lyophilized material.

Further components may also be added prior to freeze-drying e.g. as stabilizers. Preferred stabilizers for inclusion are lactose and/or sucrose and/or mannitol. Sucrose is preferred. The final vaccine may thus contain lactose and/or sucrose. A lyophilized component may also include sodium chloride. Soluble components in the lyophilized material will be retained in the composition after reconstitution.

### Non-Antigen Components of the Vaccine

As well as containing antigens, adjuvant(s) and 1-hydroxy-2-phenoxyethane, the combination vaccine produced by the process of the invention may include further components. These components may have various sources. For example, they may be present in one of the antigenic components that is mixed during the process of the invention or may be added during the process separately from the antigenic components.

To control tonicity of the final vaccine product, it is preferred to include a physiological salt, such as a sodium salt. Sodium chloride (NaCl) is preferred, which may be present in the final vaccine product at between 8-10 mg/ml e.g. about 9 mg/ml.

It is preferred not to use mercurial preservatives (e.g. thimerosal) during the process of the invention. However, the presence of trace amounts may be unavoidable if an antigen used during the process (e.g. HBsAg) has previously been treated with such a preservative. For safety, however, it is preferred that the final vaccine product contains less than 25 ng/ml mercury. More preferably, the final vaccine product contains no detectable thimerosal. This will generally be achieved by removing the mercurial preservative from an antigen preparation prior to its addition in the process of the invention e.g. using the methods of reference 30.

As well as substances such as thimerosal, other residual components from the individual antigens may also be present in trace amounts in the final vaccine produced by the process of the invention. For example, formaldehyde is used to prepare the toxoids of diphtheria, tetanus and pertussis, and so the final vaccine product may retain trace amounts of formaldehyde (e.g. less than 10 μg/ml, preferably <5 μg/ml). Detergents such as Tween 20 (polysorbate 20) or Tween 80 (polysorbate 80) may have been used during antigen (e.g. HBsAg) preparation, and so may be present in the final vaccine e.g. <10 μg/ml polysorbate 80, preferably <5 μg/ml. Media or stabilizers may have been used during poliovirus preparation (e.g. Medium 199), and these may carry through to the final vaccine. Similarly, free amino acids (e.g. alanine, arginine, aspartate, cysteine and/or cystine, glutamate, glutamine, glycine, histidine, proline and/or hydroxyproline, isoleucine, leucine, lysine, methionine, phenylalanine, serine, threonine, tryptophan, tyrosine and/or valine), vitamins (e.g. choline, ascorbate, etc.), disodium phosphate, monopotassium phosphate, calcium, glucose, adenine sulfate, phenol red, sodium acetate, potassium chloride, etc. may be retained in the final vaccine at ≦100 μg/ml. Other components from antigen preparations, such as neomycin (e.g. neomycin sulfate), polymyxin B (e.g. polymyxin B sulfate), etc. may also be present at sub-nanogram amounts per dose. A further possible component of the final vaccine which originates in the antigen preparations arises from less-than-total purification of antigens. Small amounts of B. pertussis, C. diphtheriae, C. tetani and S. cerevisiae proteins and/or genomic DNA may therefore be present. To minimize the amounts of these residual components, antigen preparations are preferably treated to remove them prior to the antigens being used in the process of the invention.

Aluminum salts are present within the final vaccine produced by the process of the invention. The total amount of aluminum, expressed in terms of Al³⁺, is preferably not more than 2 mg/ml (e.g. about 1.4 mg/ml).

During the process of the invention, dilution of components to give desired final concentrations will usually be performed with WFI (water for injection).

Where an antigenic component is substantially free from polysorbate 80 and/or polysorbate 20 then this can be achieved by ensuring that the relevant polysorbate is not used during preparation of that antigenic component, as neither polysorbate 20 nor polysorbate 80 is found naturally in C. diphtheriae, C. tetani, B. pertussis, hepatitis B virus or poliovirus.

A preservative component used in the processes of the invention is preferably antigen-free.

### Packaging of the Combination Vaccine

In typical use, the process of the invention will be used to provide bulk combination vaccine which is suitable for packaging, and then for distribution and administration. Concentrations mentioned above are typically concentrations in final packaged vaccine, and so concentrations in bulk vaccine may be higher (e.g. to be reduced to final concentrations by dilution).

The process of the invention may therefore comprise the further step of packaging the vaccine into containers for use. Suitable containers include vials and disposable syringes. These are preferably sterile.

Where the vaccine is packaged into vials, these are preferably made of glass or of a plastic material. The vial is preferably sterilized before vaccine is added to it. To avoid problems with latex-sensitive patients, vials are preferably sealed with a latex-free stopper. The vial may include a single dose of vaccine, or it may include more than one dose (a ‘multidose’ vial) e.g. 10 doses. When using a multidose vial, each dose should be withdrawn with a sterile needle and syringe under strict aseptic conditions, taking care to avoid contaminating the vial contents. Preferred vials are made of colorless glass and have metal collar on the vial's neck that is colored green and purple.

Where the vaccine is packaged into a syringe, the syringe will not normally have a needle attached to it, although a separate needle may be supplied with the syringe for assembly and use. Safety needles are preferred. 1-inch 23-gauge, 1-inch 25-gauge and ⅝-inch 25-gauge needles are typical. Syringes may be provided with peel-off labels on which the lot number and expiration date of the contents may be printed, to facilitate record keeping. The plunger in the syringe preferably has a stopper to prevent the plunger from being accidentally removed during aspiration. The syringes may have a latex rubber cap and/or plunger. Disposable syringes contain a single dose of vaccine. The syringe will generally have a tip cap to seal the tip prior to attachment of a needle, and the tip cap is preferably made of butyl rubber. If the syringe and needle are packaged separately then the needle is preferable fitted with a butyl rubber shield. Grey butyl rubber is preferred. Preferred syringes are those marketed under the trade name “Tip-Lok”™.

The combination vaccine produced by the process of the invention is preferably administered to patients in 0.5 ml doses. The process of the invention may therefore comprise the step of extracting and packaging a 0.5 ml sample of the bulk vaccine into a container. For multidose situations, multiple dose amounts will be extracted and packaged together in a single container.

The container in which the vaccine is packaged will usually then be enclosed within a box for distribution e.g. inside a cardboard box, and the box will be labeled with details of the vaccine e.g. its trade name, a list of the antigens in the vaccine (e.g. ‘Diphtheria and Tetanus Toxoids and Acellular Pertussis Adsorbed, Hepatitis B (Recombinant) and Inactivated Poliovirus Vaccine Combined’, and/or ‘DTaP HepB IPV Combined’), the presentation container (e.g. ‘Disposable Prefilled Tip-Lok Syringes’ or ‘10×0.5 ml Single-Dose Vials’), its dose (e.g. ‘each containing one 0.5 ml dose’), warnings (e.g. ‘For Pediatric Use Only’), an expiration date, etc. Each box might contain more than one packaged vaccine e.g. five or ten packaged vaccines (particularly for vials). For visual effect, preferred boxes are purple in color with a blue corner. If vaccine is contained in a syringe then the package may show a picture of the syringe.

The vaccine may be packaged together (e.g. in the same box) with a leaflet including details of the vaccine e.g. instructions for administration, details of the antigens within the vaccine, etc. The instructions may also contain warnings e.g. to keep a solution of adrenaline readily available in case of anaphylactic reaction following vaccination, etc.

### Characteristics of the Final Vaccine

For any particular antigen, vaccination guidelines (e.g. WHO guidelines) may recommend or prescribe a quantity that should be present in a final vaccine. That information (e.g. 10 μg per dose) can be combined with the volume of a vaccine dose (e.g. a 0.5 ml dose) to calculate the concentration required in the bulk vaccine for that antigen. For specific antigens listed above, preferred concentrations per dose in the final vaccine to be administered to a patient are as follows:

| Antigen | Concentration per dose* | |
|---|---|---|
| Diphtheria toxoid | 25 | Lf |
| Tetanus toxoid | 10 | Lf |
| Pertussis toxoid | 25 | μg |
| Filamentous hemagglutinin | 25 | μg |
| Pertactin | 8 | μg |
| HBsAg | 10 | μg |
| Type 1 poliovirus | 40 | DU |
| Type 2 poliovirus | 8 | DU |
| Type 3 poliovirus | 32 | DU |

*These doses are given ± 10%

The concentration of individual antigens in the bulk vaccine, the dilutions required before addition of antigens to the bulk vaccine, and any dilution required from bulk to final vaccine can be calculated accordingly, and thus the process of the invention may performed to produce a final combination vaccine which contains antigens at these concentrations per dose. Any dilution will typically use water for injection (WFI).

The final vaccine is preferably sterile. The final vaccine is preferably non-pyrogenic e.g. it contains <1 EU (endotoxin unit; 1 EU is equal to 0.2 ng FDA reference standard Endotoxin EC-2 ‘RSE’) per dose, and preferably <0.1 EU per dose.

The final vaccine is preferably gluten free.

The packaged vaccine is preferably sterile.

The pH of the final vaccine is preferably between 6 and 8 e.g. between 6.5 and 7.5. A process of the invention may therefore include a step of adjusting the pH of the bulk vaccine prior to packaging.

The packaged vaccine is preferably a turbid white suspension.

The packaged vaccine is preferably stored at between 2° C. and 8° C. It should not be frozen.

### The Mixing Process

The process of the involves mixing components. These components may be added in any suitable order, and the order may depend on various factors. For example, an antigen may be added early in the process if it is desired that it should adsorb to a component which is already present in a nascent vaccine mixture. Conversely, it may be added late in the process, after other antigens have been added, if it is desired that the adsorption capacity of the components of the nascent vaccine mixture should be minimal.

Antigens may or may not be adsorbed to adjuvant prior to being used in a process of the invention. In particular, it is preferred that pertussis antigens and HBsAg are adsorbed prior to addition, whereas poliovirus is not adsorbed prior to addition.

Non-antigen components may be added as part of an antigenic component, or may be added separately from the antigens. Adjuvants are generally added with antigens. Other components, such as antimicrobials, NaCl, etc. may be added with antigens or may be added separately.

Antigens are preferably mixed into a sterile sodium chloride solution. The sodium chloride solution preferably consists of pharmaceutical grade sodium chloride dissolved in water for injection.

### Aluminum Adjuvants

As described above, the antigens that are present in the final vaccine may be adsorbed to aluminum adjuvants prior to being used in the process of the invention (‘pre-adsorbed’).

Aluminum adjuvants currently in use are typically referred to either as “aluminum hydroxide” or as “aluminum phosphate” adjuvants. These are names of convenience, however, as neither is a precise description of the actual chemical compound which is present (e.g. see chapter 9 of reference 72). The invention can use any of the “hydroxide” or “phosphate” adjuvants that are in general use as adjuvants.

The adjuvants known as “aluminum hydroxide” are typically aluminum oxyhydroxide salts, which are usually at least partially crystalline. Aluminum oxyhydroxide, which can be represented by the formula AlO(OH), can be distinguished from other aluminum compounds, such as aluminum hydroxide Al(OH)₃, by infrared (IR) spectroscopy, in particular by the presence of an adsorption band at 1070 cm⁻¹ and a strong shoulder at 3090-3100 cm⁻¹ [chapter 9 of ref. 72].

The adjuvants known as “aluminum phosphate” are typically aluminum hydroxyphosphates, often also containing a small amount of sulfate. They may be obtained by precipitation, and the reaction conditions and concentrations during precipitation influence the degree of substitution of phosphate for hydroxyl in the salt. Hydroxyphosphates generally have a PO₄/Al molar ratio between 0.3 and 0.99. Hydroxyphosphates can be distinguished from strict AlPO₄ by the presence of hydroxyl groups. For example, an IR spectrum band at 3164 cm⁻¹ (e.g. when heated to 200° C.) indicates the presence of structural hydroxyls [chapter 9 of ref. 72].

The invention provides a process for manufacturing a combination vaccine, wherein:
(a) the vaccine comprises a diphtheria toxoid, a tetanus toxoid, a pertussis toxoid, filamentous hemagglutinin, pertactin, a hepatitis B surface antigen and poliovirus antigens;
(b) the process comprises the steps of mixing (i) a first antigenic component that comprises (1) a diphtheria toxoid adsorbed to an aluminum hydroxide adjuvant (2) a tetanus toxoid adsorbed to an aluminum hydroxide adjuvant and (3) a 1-hydroxy-2-phenoxyethane preservative, (ii) a second antigenic component that comprises a hepatitis B surface antigen adsorbed to an aluminum phosphate adjuvant, (iii) a third antigenic component that comprises one or more poliovirus antigens not adsorbed to an aluminum salt, and (iv) a fourth antigenic component that comprises pertactin adsorbed to an aluminum hydroxide adjuvant and, optionally, comprises filamentous hemagglutinin and/or a pertussis toxoid.

In this process, at least one (i.e. 1, 2 or 3) of the second, third and fourth antigenic components may be substantially free from 1-hydroxy-2-phenoxyethane.

The invention also provides a process for manufacturing a combination vaccine, wherein:
(a) the vaccine comprises a diphtheria toxoid, a tetanus toxoid, a pertussis toxoid, filamentous hemagglutinin, pertactin, a hepatitis B surface antigen and poliovirus antigens;
(b) the process comprises the steps of mixing (i) a first antigenic component that comprises (1) a diphtheria toxoid adsorbed to an aluminum hydroxide adjuvant (2) a tetanus toxoid adsorbed to an aluminum hydroxide adjuvant and (3) a 1-hydroxy-2-phenoxyethane preservative, (ii) a second antigenic component that comprises a hepatitis B surface antigen adsorbed to an aluminum phosphate adjuvant, (iii) a third antigenic component that comprises one or more poliovirus antigens not adsorbed to an aluminum salt, (iv) a fourth antigenic component that comprises pertactin adsorbed to an aluminum hydroxide adjuvant and, optionally, comprises filamentous hemagglutinin and/or a pertussis toxoid, and (v) a preservative component that comprises 1-hydroxy-2-phenoxyethane;
(c) the preservative component is substantially free from any diphtheria toxoid, tetanus toxoid, acellular pertussis antigens, hepatitis B surface antigen and poliovirus antigens.

In this process, each of the second, third and fourth antigenic components may be substantially free from 1-hydroxy-2-phenoxyethane.

### Administration of the Vaccine

The final combination vaccine produced by the process of the invention is suitable for administration to humans, and in particular to children. A typical dosage schedule for the vaccine, or order to have full efficacy, will involve administering more than one dose in a primary immunization schedule. A typical primary schedule will involve three doses, given at intervals of about 6 to 8 weeks, with the first dose being given to a child aged between 6 and 9 weeks of age. The vaccine may also be used to complete the primary immunization schedule of a different vaccine.

The final combination vaccine produced by the process of the invention should be administered by intramuscular injection. Preferred sites for injection are the anterolateral thigh or the deltoid muscle of the upper arm.

Vaccines prepared by the invention may be administered at substantially the same time as, but at a separate injection from, a pneumococcal conjugate vaccine, such as PREVNAR™.

A 5-valent vaccine including diphtheria toxoid, tetanus toxoid, acellular pertussis antigen(s), HBsAg and poliovirus antigen(s) may be administered at substantially the same time as, but at a separate injection from, for instance: (1) a H. influenzae type b vaccine; (2) a combination vaccine comprising a H. influenzae type b conjugate and one or more N. meningitidis conjugate(s) e.g. a mixture of Hib and MenC conjugates, or a mixture of Hib, MenC and MenY conjugates, etc.

Thus a patient may receive a first vaccine and a second vaccine, wherein the first vaccine is a vaccine prepared by the processes of the invention. The second vaccine will be for protecting the patient against a disease which the first vaccine does not protect against. For instance, if the first vaccine does not include a H. influenzae type b conjugate then such a conjugate can be included in the second vaccine. Similarly, the second vaccine may include: a mixture of Hib and MenC conjugates; a mixture of Hib, MenC and MenY conjugates; one or more pneumococcal conjugates; etc. The first and second vaccines will typically be administered to the patient at substantially the same time as each other e.g. during the same medical consultation or visit to a healthcare professional.

As the final combination vaccine produced by the process of the invention contains an aluminum-based adjuvant, settling of components may occur during storage. The vaccine should therefore be shaken prior to administration to a patient. The shaken vaccine will be a turbid white suspension.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x±10%.

Where a component is described as being “adsorbed” to an adjuvant, it is preferred that at least 50% (by weight) of that antigen is adsorbed e.g. 50%, 60%, 70%, 80%, 90%, 95%, 98% or more. If a component is totally adsorbed then none should detectable in the supernatant of a composition after centrifugation.

### Example

The following components were prepared:
diphtheria toxoid concentrate.
tetanus toxoid concentrate.
aluminum hydroxide suspension, with a Al⁺⁺⁺ concentration of 15 g/l.
a solution of 1-hydroxy-2-phenoxyethane at 13 g/l.
a saturated sodium chloride solution.
   All components were sterile and were prepared using WFI.

118.3×10⁶ Lf of the diphtheria toxoid concentrate was mixed in a 50 liter vessel with 22 liters of the aluminum hydroxide suspension. The contents were mixed for at least 30 minutes at 80-100 rpm with a magnetic stirrer. This process gives a pre-adsorbed diphtheria toxoid component.

47.5×10⁶ Lf of the tetanus toxoid concentrate was mixed in a 50 liter vessel with 28 liters of the aluminum hydroxide suspension. The contents were mixed for at least 30 minutes at 80-100 rpm with a magnetic stirrer. This process gives a pre-adsorbed tetanus toxoid component.

Further aluminum hydroxide suspension was placed in a new mixing vessel. The pre-adsorbed diphtheria toxoid was added, and then the pre-adsorbed tetanus toxoid was added. The 1-hydroxy-2-phenoxyethane solution was then added, followed by 14-18 liters of the sodium chloride solution. Proportions were chosen to give a final Al⁺⁺⁺ concentration between 2.1 and 2.6 mg/ml, a final sodium chloride concentration of between 8 and 9 mg/ml, a final 1-hydroxy-2-phenoxyethane concentration of 5 mg/ml, a final diphtheria toxoid concentration of 167 Lf/ml and a final tetanus toxoid concentration of 67 Lf/ml. After further mixing, the pH of the mixture was adjusted to be between 6.0 and 6.3. This material is an adsorbed concentrate of diphtheria and tetanus toxoids for use in manufacturing combination vaccines (“DT bulk”).

Pertussis toxoid, FHA and pertactin are purified, adsorbed to an aluminum hydroxide adjuvant and combined to give bulk acellular pertussis antigen. This bulk acellular pertussis antigen can be combined with the DT bulk to give a D-T-Pa vaccine.

Yeast-expressed HBsAg is purified and adsorbed to an aluminum phosphate adjuvant to give bulk HBV antigen. This bulk HBV antigen can be combined with the DT bulk and the acellular pertussis bulk to give a D-T-Pa-HBsAg vaccine.

Polioviruses (Mahoney strain, MEF-1 strain and Saukett strain) are grown separately on Vero cells and virions are purified then inactivated. The three viruses are then mixed to give an inactivated poliovirus vaccine (IPV) bulk, without adjuvant. This IPV bulk can be combined with the DT bulk, the acellular pertussis bulk and the HBV bulk to give a 5-valent D-T-Pa-HBsAg-IPV vaccine.

During manufacture of the 5-valent product, a sterile solution of 1-hydroxy-2-phenoxyethane is added as a separate component, to give a bulk vaccine with the following content:

| Antigen | Concentration per ml | |
|---|---|---|
| Diphtheria toxoid | 50 | Lf |
| Tetanus toxoid | 20 | Lf |
| Inactivated pertussis toxin | 50 | μg |
| Filamentous hemagglutinin | 50 | μg |
| Pertactin | 16 | μg |
| HBsAg | 20 | μg |
| Type 1 poliovirus | 80 | DU |
| Type 2 poliovirus | 16 | DU |
| Type 3 poliovirus | 64 | DU |
| 1-hydroxy-2-phenoxyethane | 5 | mg |
| Sodium chloride | 9 | mg |

To give these final concentrations, bulks can be diluted and mixed using a sterile sodium chloride solution. The bulk 5-valent vaccine can then be packaged into 1 ml glass syringes, including a unit dose of 0.5 ml in each syringe. The packaged vaccines can then be distributed for use by physicians. The 5-valent vaccine can be used on its own, or can be used to reconstitute other lyophilized vaccines e.g. a lyophilized Hib conjugate, or a lyophilized mixture of a Hib conjugate and a serogroup C meningococcus conjugate, or a lyophilized mixture of a Hib conjugate, a serogroup C meningococcus conjugate, and a serogroup Y meningococcus conjugate.

It will be understood that the invention has been described by way of example only and modifications may be made whilst remaining within the scope and spirit of the invention.

### REFERENCES

### The Contents of which are Hereby Incorporated by Reference

[1] Murdoch et al. (2003) Drugs 63(23):2625-49.
[2] MMWR Morb Mortal Wkly Rep (1997) 46(5):107-9.
[3] Wellington & Goa (2003) Drugs 63(19):2107-26.
[4] Patel & Wagstaff (1996) Drugs 52:254-75.
[5] MMWR Morb Mortal Wkly Rep (1996) 45(45):993-5.
[6] Yeh et al. (2001) Pediatr Infect Dis J 20:973-80.
[7] Curran & Goa (2003) Drugs 63(7):673-82.
[8] Mallet et al. (2000) Pediatr. Infect. Dis. J 19:1119-1127.
[9] Lowe & Southern (1994) Lett Appl Microbiol 18:115-116.
[10] Komatsu et al. (2002) J Health Sci 48:89-92.
[11] Vogt et al. (1998) Contact Dermatitis 38:50-51.
[12] MuBhoff et al. (2000) Arch Toxicol 74:284-287.
[13] http://www.vaccinetruth.org/2-phenoxyethanol.htm
[14] Vaccines. (eds. Plotkin & Orenstein). 4th edition, 2004, ISBN: 0-7216-9688-0.
[15] National Institute for Biological Standards and Control; Potters Bar, UK. www.nibsc.ac.uk
[16] Sesardic et al. (2001) Biologicals 29:107-22.
[17] NIBSC code: 98/560.
[18] Sesardic et al. (2002) Biologicals 30:49-68.
[19] NIBSC code: 98/552.
[20] Module 1 of WHO's The immunological basis for immunization series (Galazka).
[21] NIBSC code: 69/017.
[22] NIBSC code: DIFT.
[23] NIBSC code: TEFT.
[24] European patent 0460716; U.S. Pat. No. 5,349,059.
[25] Crabeel et al. (1983) Proc Natl Acad Sci USA 78:5026-30.
[26] Cabezón et al. (1984) Proc Natl Acad Sci USA 81:6594-8.
[27] van der Straten et al. (1986) DNA 5:129-36.
[28] Harford et al. (1987) Postgraduate Medical Journal 63(suppl 2):65-70.
[29] U.S. Pat. No. 6,013,264
[30] WO03/066094.
[31] Module 6 of WHO's The immunological basis for immunization series (Robertson)
[32] Rappuoli et al. (1991) TIBTECH 9:232-238.
[33] Anonymous (January 2002) Research Disclosure, 453077.
[34] Anderson (1983) Infect Immun 39(1):233-238.
[35] Anderson et al. (1985) J Clin Invest 76(1):52-59.
[36] EP-A-0372501.
[37] EP-A-0378881.
[38] EP-A-0427347.
[39] WO93/17712
[40] WO94/03208.
[41] WO98/58668.
[42] EP-A-0471177.
[43] WO91/01146
[44] Falugi et al. (2001) Eur J Immunol 31:3816-24.
[45] Baraldo et al. (2004) Infect Immun 72:4884-87.
[46] EP-A-0594610.
[47] WO00/56360.
[48] WO02/091998.
[49] Kuo et al. (1995) Infect Immun 63:2706-13.
[50] WO01/72337
[51] WO00/61761.
[52] WO2004/041157.
[53] WO2007/000327.
[54] WO2007/000343.
[55] WO2007/000342.
[56] WO2007/000341.
[57] WO02/080965.
[58] WO02/058737.
[59] WO03/007985.
[60] WO03/080678.
[61] Glode et al. (1979) J Infect Dis 139:52-56
[62] WO94/05325; U.S. Pat. No. 5,425,946.
[63] Arakere & Frasch (1991) Infect. Immun. 59:4349-4356.
[64] Michon et al. (2000) Dev. Biol. 103:151-160.
[65] Rubinstein & Stein (1998) J. Immunol. 141:4357-4362.
[66] WO2005/033148
[67] WO2005/000347.
[68] WO02/058737.
[69] WO03/007985.
[70] WO2007/000314.
[71] WO2007/000322.
[72] Vaccine Design: The Subunit and Adjuvant Approach (eds. Powell & Newman) Plenum Press 1995 (ISBN 0-306-44867-X).

1. A process for manufacturing a combination vaccine, wherein:
(a) the vaccine comprises a diphtheria toxoid, a tetanus toxoid, and at least one further antigen; and
(b) the process involves mixing a first component and a second component, wherein
the first component comprises a diphtheria toxoid, a tetanus toxoid and 1-hydroxy-2-phenoxyethane, and
the second component comprises said at least one further antigen.

2. The process of claim 1, wherein the second component is substantially free from any diphtheria toxoid or tetanus toxoid.

3. The process of claim 1, wherein the diphtheria and tetanus toxoids in the first component are adsorbed to an aluminum hydroxide adjuvant.

4. The process of claim 1, wherein the first component is free from mercury.

5. The process of claim 1, wherein the second component includes one or more acellular pertussis antigens.

6. The process of claim 1, wherein the second component includes one or more poliovirus antigens.

7. The process of claim 1, wherein the second component includes a conjugate of the capsular saccharide antigen from Haemophilus influenzae type B.

8. The process of claim 1, wherein the second component includes a hepatitis B surface antigen (‘HbsAg’).

9. The process of claim 1, wherein the combination vaccine includes diphtheria toxoid, tetanus toxoid, hepatitis B surface antigen, poliovirus antigens and acellular pertussis antigens.

10. The process of claim 1, wherein the second component includes 1-hydroxy-2-phenoxyethane.

11. The process of claim 1, wherein the second component is substantially free of 1-hydroxy-2-phenoxyethane.

12. The process of claim 11, wherein the process includes a further step of mixing the first and second components with a source of 1-hydroxy-2-phenoxyethane.

13. The process of claim 1, wherein the first component contains diphtheria toxoid and tetanus toxoid in a ratio (measured in Lf units) of between 2:1 and 3:1.

14. The process of claim 1, wherein the first component contains diphtheria toxoid and tetanus toxoid in a ratio (measured in Lf units) of 2.5:1.

15. The process of claim 1, wherein the first component contains between 2.5 mg and 3.5 mg of 1-hydroxy-2-phenoxyethane for every 100 Lf of diphtheria toxoid.

16. The process of claim 1, wherein the first component contains between 7 mg and 8 mg of 1-hydroxy-2-phenoxyethane for every 100 Lf of tetanus toxoid.

17. A process for manufacturing a combination vaccine, wherein:
(a) the vaccine comprises a diphtheria toxoid, a tetanus toxoid, an acellular pertussis antigen, a hepatitis B surface antigen and poliovirus antigens;
(b) the process comprises the steps of mixing:
(i) a first antigenic component that comprises both a diphtheria toxoid and a tetanus toxoid,
(ii) a second antigenic component that comprises a hepatitis B surface antigen,
(iii) a third antigenic component that comprises one or more poliovirus antigens, and
(iv) a fourth antigenic component that comprises one or more acellular pertussis antigens,
(c) the first antigenic component also comprises 1-hydroxy-2-phenoxyethane; and
(d) at least one of the second, third and fourth antigenic components is substantially free from 1-hydroxy-2-phenoxyethane.

18. The process of claim 17, wherein none of the second, third and fourth antigenic components includes 1-hydroxy-2-phenoxyethane.

19. The process of claim 18, wherein additional 1-hydroxy-2-phenoxyethane is added separately from the first to fourth antigenic components.

20. A process for manufacturing a combination vaccine, wherein:
(a) the vaccine comprises a diphtheria toxoid, a tetanus toxoid, an acellular pertussis antigen, a hepatitis B surface antigen and poliovirus antigens;
(b) the process comprises the steps of mixing:
(i) a first antigenic component that comprises both a diphtheria toxoid and a tetanus toxoid,
(ii) a second antigenic component that comprises a hepatitis B surface antigen,
(iii) a third antigenic component that comprises one or more poliovirus antigens,
(iv) a fourth antigenic component that comprises one or more acellular pertussis antigens, and
(v) a preservative component that comprises 1-hydroxy-2-phenoxyethane;
(c) the first antigenic component also comprises 1-hydroxy-2-phenoxyethane;
(d) each of the second, third and fourth antigenic components is substantially free from 1-hydroxy-2-phenoxyethane; and
(e) the preservative component is substantially free from any diphtheria toxoid, tetanus toxoid, acellular pertussis antigens, hepatitis B surface antigen and poliovirus antigens.

21. The process of claim 1, wherein the combination vaccine includes about 5 mg/ml of 1-hydroxy-2-phenoxyethane.

22. The process of claim 1, wherein the combination vaccine has antigens present at the following concentrations per milliliter (±10%): 50 Lf diphtheria toxoid; 20 Lf tetanus toxoid; 50 μg inactivated pertussis toxin; 50 μg filamentous hemagglutinin; 16 μg pertactin; 20 μg HBsAg; 80 DU Type 1 poliovirus; 16 DU Type 2 poliovirus; and 64 DU Type 3 poliovirus.

23. The process of claim, wherein the HBsAg is yeast-expressed.

24. The process of claim 23, wherein the HBsAg is non-glycosylated.

25. The process of claim 24, wherein the HBsAg is in the form of particles including a lipid matrix comprising phospholipids, phosphatidylinositol and polysorbate 20.

26. The process of claim 23, wherein the HBsAg is expressed in yeast (1) under the control of an upstream promoter from a glyceraldehyde-3-phosphate dehydrogenase gene; and/or (2) with a downstream ARG3 transcription terminator.

27. The process of claim 8, wherein the HBsAg has amino acid sequence SEQ ID NO: 1.

28. The process of claim 8, wherein the HBsAg is adsorbed to an aluminum phosphate adjuvant.

29. The process of claim 8, wherein the poliovirus antigens include antigens from a poliovirus Type 1 strain, a poliovirus Type 2 and a poliovirus Type 3 strain.

30. The process of claim 8, wherein the acellular pertussis antigen includes detoxified pertussis toxin, filamentous hemagglutinin and pertactin.

31. The process of claim 30, wherein pertactin is adsorbed onto an aluminum hydroxide adjuvant.

32. The process of claim 1, wherein the vaccine is packaged as an aqueous component of a kit, and wherein the kit also includes a lyophilized antigenic component.

33. The process of claim 32, wherein the lyophilized antigenic component includes one or more conjugates of a bacterial capsular saccharide.

34. The process of claim 33, wherein the lyophilized antigenic component includes a conjugate of a capsular saccharide antigen from Haemophilus influenzae type B.

35. The process of claim 33, wherein the lyophilized antigenic component includes a conjugate of a capsular saccharide antigen from a Neisseria meningitidis.

36. The process of claim 35, wherein the lyophilized antigenic component includes a conjugate of a capsular saccharide antigen from serogroup C of Neisseria meningitidis and/or a conjugate of a capsular saccharide antigen from serogroup Y of Neisseria meningitidis.

37. The process of claim 33, wherein one or more of the conjugates includes a tetanus toxoid carrier protein.

38. The process of claim 33, wherein one or more of the conjugates has saccharide:protein ratio (w/w) of between 1:5 and 5:1.

39. A process for preparing a two-container combination vaccine, comprising the following steps:
preparing a combination vaccine according to claim 1, but wherein the said one or more antigens does not include a H. influenzae type B antigen;
packaging said combination vaccine in a first container;
preparing a freeze-dried H. influenzae type B antigen;
packaging said freeze-dried H. influenzae type B antigen in a second container; and
packaging the first container and second container together in a kit.

40. The process of claim 39, wherein the second container also contains a conjugated N. meningitidis serogroup C saccharide antigen.

41. The process of claim 40, wherein the contents of the second container are aluminium-free.

42. The process of claim 40, wherein the contents of the second container are adjuvant-free.

43. A process for manufacturing a combination vaccine, wherein:
(a) the vaccine comprises a diphtheria toxoid, a tetanus toxoid, a pertussis toxoid, filamentous hemagglutinin, pertactin, a hepatitis B surface antigen and poliovirus antigens;
(b) the process comprises the steps of mixing
(i) a first antigenic component that comprises
(1) a diphtheria toxoid adsorbed to an aluminum hydroxide adjuvant
(2) a tetanus toxoid adsorbed to an aluminum hydroxide adjuvant and
(3) a 1-hydroxy-2-phenoxyethane preservative,
(ii) a second antigenic component that comprises a hepatitis B surface antigen adsorbed to an aluminum phosphate adjuvant,
(iii) a third antigenic component that comprises one or more poliovirus antigens not adsorbed to an aluminum salt, and
(iv) a fourth antigenic component that comprises pertactin adsorbed to an aluminum hydroxide adjuvant and, optionally, comprises filamentous hemagglutinin and/or a pertussis toxoid.

44. The process of claim 43, wherein at least one of the second, third and fourth antigenic components is substantially free from 1-hydroxy-2-phenoxyethane.

45. A process for manufacturing a combination vaccine, wherein:
(a) the vaccine comprises a diphtheria toxoid, a tetanus toxoid, a pertussis toxoid, filamentous hemagglutinin, pertactin, a hepatitis B surface antigen and poliovirus antigens;
(b) the process comprises the steps of mixing
(i) a first antigenic component that comprises
(1) a diphtheria toxoid adsorbed to an aluminum hydroxide adjuvant
(2) a tetanus toxoid adsorbed to an aluminum hydroxide adjuvant and
(3) a 1-hydroxy-2-phenoxyethane preservative,
(ii) a second antigenic component that comprises a hepatitis B surface antigen adsorbed to an aluminum phosphate adjuvant,
(iii) a third antigenic component that comprises one or more poliovirus antigens not adsorbed to an aluminum salt,
(iv) a fourth antigenic component that comprises pertactin adsorbed to an aluminum hydroxide adjuvant and, optionally, comprises filamentous hemagglutinin and/or a pertussis toxoid, and
(v) a preservative component that comprises 1 hydroxy-2-phenoxyethane;
(c) the preservative component is substantially free from any diphtheria toxoid, tetanus toxoid, acellular pertussis antigens, hepatitis B surface antigen and poliovirus antigens.

46. The process of claim 45, wherein each of the second, third and fourth antigenic components is substantially free from 1 hydroxy-2-phenoxyethane.

47. A process for immunizing a patient, comprising the steps of administering to the patient:
(i) a first vaccine prepared by the process of any preceding claim, provided that the vaccine does not include a H. influenzae type b conjugate; and
(ii) a second vaccine comprising a H. influenzae type b conjugate, wherein the first and second vaccines are administered to the patient at substantially the same time as each other.

48. The process of claim 47, wherein the second vaccine comprises a conjugate of a capsular saccharide from N. meningitidis serogroup C, but the first vaccine does not.

49. The process of claim 47, wherein the second vaccine comprises a conjugate of a capsular saccharide from N. meningitidis serogroup Y, but the first vaccine does not.

50. The process of claim 48, wherein at least one of the conjugates has a tetanus toxoid carrier protein.

51. The process of claim 50, wherein each of the conjugates has a tetanus toxoid carrier protein.
